(19) United States
(12) Patent Application Publication
(10) Pub. No.: US 2025/0352170 A1
(43) Pub. Date: Nov. 20, 2025

(54) ULTRASOUND DIAGNOSIS APPARATUS AND STORAGE MEDIUM

(71) Applicant: Canon Medical Systems Corporation, Otawara-shi (JP)

(72) Inventors: Yasunori HONJO, Utsunomiya (JP); Takeshi FUKASAWA, Nasushiobara (JP)

(73) Assignee: Canon Medical Systems Corporation, Otawara-shi (JP)

(21) Appl. No.: 19/188,241

(22) Filed: Apr. 24, 2025

(30) Foreign Application Priority Data
May 14, 2024 (JP) 2024-078893

(51) Int. Cl. A61B 8/08; A61B 8/00; A61B 8/12

(52) U.S. Cl. CPC A61B 8/0841; A61B 8/12; A61B 8/463; A61B 8/466; A61B 8/469; A61B 8/483

(57) ABSTRACT

According to one embodiment, an ultrasound diagnosis apparatus includes processing circuitry. The processing circuitry is configured to generate a first ultrasound image of a subject into which a medical tool has been inserted by executing, via an ultrasound probe, non-3D scanning of the subject in which at least one scanning surface is formed. The processing circuitry is configured to generate a second ultrasound image containing the medical tool by executing 3D scanning involving the first scanning. The processing circuitry is configured to estimate, based on the second ultrasound image, an angle of a first scanning surface of an ultrasound probe with which a first ultrasound image containing the medical tool can be generated. The processing circuitry is configured to switch from the non-3D scanning to execute the 3D scanning during image displaying in the non-3D scanning, and outputs the estimated angle of the first scanning surface.

### CROSS-REFERENCE TO RELATED APPLICATION

This application is based upon and claims the benefit of priority from prior Japanese Patent Application No. 2024-078893, filed May 14, 2024, the entire contents of which are incorporated herein by reference.

### FIELD

Embodiments described herein relate generally to an ultrasound diagnosis apparatus and a storage medium.

### BACKGROUND

Conventionally, an ultrasound diagnosis apparatus for capturing an image of a medical tool used in a catheter intervention for structural heart diseases (SHD) has been known. In this type of ultrasound diagnosis apparatus, an ultrasound image containing a medical tool is generated based on an output from an ultrasound probe, and the generated ultrasound image is displayed on a display. In a catheter intervention, the ultrasound diagnosis apparatus is configured, for example, to generate and display, based on an output from a transesophageal probe, an ultrasound image of the heart of a subject into which a medical tool such as a guide wire or a device (a clip, an artificial valve, or the like) has been inserted, in such a manner that the ultrasound image contains a tip end part of a medical tool. At this time, from the perspective of frame rate and resolution, the ultrasound diagnosis apparatus displays mainly a two-dimensional ultrasound image containing the tip end part of the medical tool, for example.

If, however, the operator has lost sight of the tip end part of the medical tool, or is observing a portion other than the tip end part such as the heart valve, the ultrasound diagnosis apparatus displays a two-dimensional ultrasound image not containing the tip end part of the medical tool. After the two-dimensional ultrasound image is displayed, the operator searches for the tip end part on the two-dimensional ultrasound image to observe the tip end part again. For example, the operator performs an operation of adjusting an angle of a scanning surface of the transesophageal probe while observing the two-dimensional ultrasound image. Alternatively, the operator switches from a 2D mode of generating a two-dimensional ultrasound image to a 3D mode of executing 3D scanning, and observes a three-dimensional ultrasound image generated by executing the 3D scanning. The operator confirms an overall image of a region including the tip end part of the medical tool by observing the three-dimensional ultrasound image, and performs an operation of adjusting the angle of the scanning surface by switching to the 2D mode again.

Such an ultrasound diagnosis apparatus is not particularly problematic, but leaves room for improvement in that the operation of searching for the tip end part of the medical tool could be burdensome for some operators, according to the present inventors' study. There is thus a demand for a technique of supporting the operator's operation of searching for a medical tool on an ultrasound image.

### BRIEF DESCRIPTION OF THE DRAWINGS

FIG. 1 shows an example of a configuration of an ultrasound diagnosis apparatus according to a first embodiment.

FIG. 2 illustrates position information of an ultrasound probe according to the first embodiment.

FIG. 3 illustrates a trained model according to the first embodiment.

FIG. 4 is a timing chart illustrating an example of an operation in the ultrasound diagnosis apparatus according to the first embodiment.

FIG. 5 shows an example of a two-dimensional ultrasound image generated by the ultrasound diagnosis apparatus according to the first embodiment.

FIG. 6 is a flowchart illustrating an example of an operation in the ultrasound diagnosis apparatus according to the first embodiment.

FIG. 7 shows an example of a three-dimensional ultrasound image generated by the ultrasound diagnosis apparatus according to the first embodiment.

FIG. 8 shows an example of a two-dimensional ultrasound image generated by the ultrasound diagnosis apparatus according to the first embodiment.

FIG. 9 is a flowchart illustrating an example of an operation in an ultrasound diagnosis apparatus according to a modification of the first embodiment.

FIG. 10 shows an example of a configuration of an ultrasound diagnosis apparatus according to a second embodiment.

FIG. 11 is a flowchart illustrating an example of an operation in the ultrasound diagnosis apparatus according to the second embodiment.

FIG. 12 shows an example of a configuration of an ultrasound diagnosis apparatus according to a third embodiment.

FIG. 13 is a flowchart illustrating an example of an operation in the ultrasound diagnosis apparatus according to the third embodiment.

FIG. 14 is a flowchart illustrating an example of an operation in an ultrasound diagnosis apparatus according to a modification of the third embodiment.

FIG. 15 shows an example of a configuration of an ultrasound diagnosis apparatus according to a fourth embodiment.

FIG. 16 is a flowchart illustrating an example of an operation in the ultrasound diagnosis apparatus according to the fourth embodiment.

FIG. 17 is a flowchart illustrating an example of an operation in an ultrasound diagnosis apparatus according to a modification of the fourth embodiment.

### DETAILED DESCRIPTION

In general, according to one embodiment, an ultrasound diagnosis apparatus includes processing circuitry. The processing circuitry is configured to generate a first ultrasound image of a subject into which a medical tool has been inserted by executing, via an ultrasound probe, non-3D scanning of the subject in which at least one scanning surface is formed. The processing circuitry is configured to generate a second ultrasound image containing the medical tool by executing 3D scanning involving the first scanning. The processing circuitry is configured to estimate, based on the second ultrasound image, an angle of a first scanning surface of an ultrasound probe with which a first ultrasound image containing the medical tool can be generated. The processing circuitry is configured to switch from the non-3D scanning to execute the 3D scanning during image displaying in the non-3D scanning, and outputs the estimated angle of the first scanning surface.

Hereinafter, embodiments of an ultrasound diagnosis apparatus will be described with reference to the accompanying drawings. In the description that follows, substantially the same elements shown in different drawings will be referred to by the same reference numerals to omit redundant explanations.

### First Embodiment

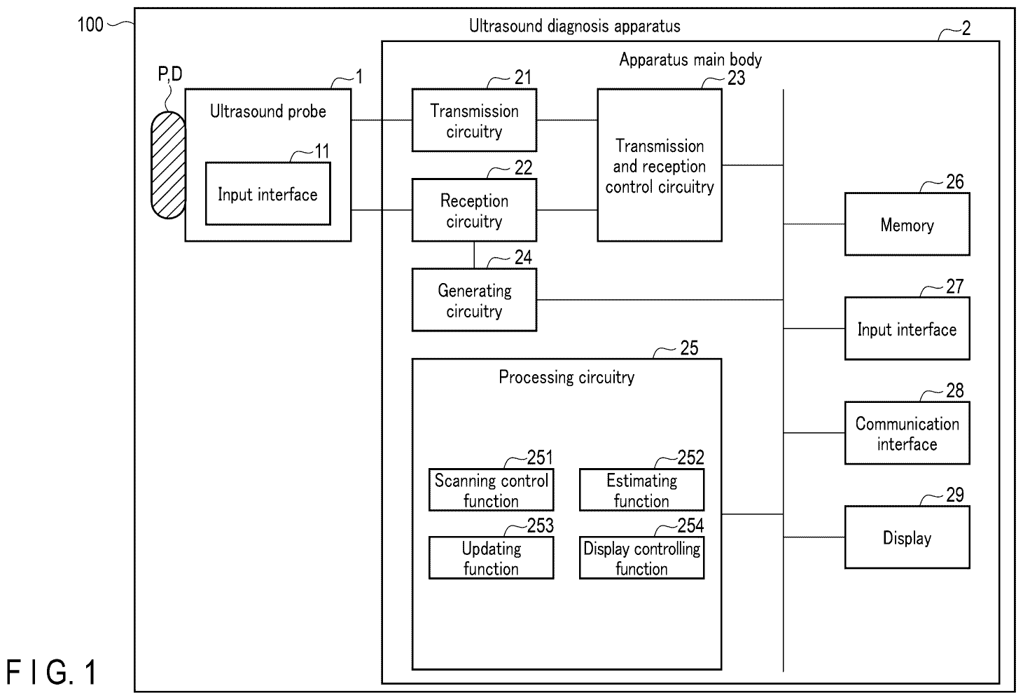

FIG. 1 shows a configuration example of an ultrasound diagnosis apparatus 100 according to a first embodiment. As shown in FIG. 1, the ultrasound diagnosis apparatus 100 includes an ultrasound probe 1 and an apparatus main body 2. The ultrasound probe 1 is detachably connected to the apparatus main body 2.

The ultrasound diagnosis apparatus 100 is configured, for example, to capture an image of a tip end part of a medical tool D used in a catheter intervention for a structural heart disease, as well as a portion other than the tip end part such as the heart valve. Examples of the medical tool D include a clip (e.g., MitraClip™) placed at the mitral valve via percutaneous mitral valve clipping, which is a type of catheter intervention. Examples of the medical tool D also include an artificial valve with which the aortic valve has been replaced via transcatheter aortic valve implantation (TAVI), which is also a type of catheter intervention. Examples of the medical tool D further include a catheter and a guide wire used in a catheter intervention. In a catheter intervention, the ultrasound diagnosis apparatus 100 is configured to generate and display, based on an output from the ultrasound probe 1, an ultrasound image of the heart of a subject into which the medical tool D has been inserted, in such a manner that the ultrasound image contains the tip end part of the medical tool D. It is to be noted that a clip or an artificial valve is an example of a first medical tool D or a second medical tool D. A clip or an artificial valve may be referred to as a “device”. A catheter or a guide wire may also be an example of the first medical tool D or the second medical tool D.

The ultrasound probe 1 is, for example, an endocavity ultrasound probe configured to be inserted into a subject's body cavity. Specifically, the ultrasound probe 1 is, for example, a transesophageal probe inserted into the esophagus of the subject and configured to receive a reflected-wave signal related to the medical tool D from the esophagus. The ultrasound probe 1 includes a tip end part, a bending part, a guide tube part, a handle part, a cable part, and a connector part, which are not illustrated. The tip end part includes, for example, a plurality of piezoelectric transducers aligned one-dimensionally, matching layers provided for the piezoelectric transducers, and a backing material for preventing ultrasound waves from propagating rearward from the piezoelectric transducers. The piezoelectric transducers are configured to generate an ultrasound wave based on a drive signal supplied thereto from the transmission circuitry 21 of the apparatus main body 2 via the cable part. The piezoelectric transducers are configured, for example, to be rotatable in response to a motion from a motor (not illustrated) contained in the ultrasound probe 1. The bending part is connected to the tip end part, and has bending properties. The guide tube part is connected to the bending part, and is configured to be inserted into the subject's body cavity for capturing an ultrasound image of the subject. A handle part is connected to the guide tube part, and is gripped by the operator. The handle part includes an input interface 11. The cable part transmits a signal exchanged between the apparatus main body 2 and the tip end part and the handle part. The connector part electrically connects the ultrasound probe 1 and the apparatus main body 2. It is to be noted that the ultrasound probe 1 is not limited to a configuration in which a plurality of piezoelectric transducers are one-dimensionally aligned, and may be, for example, a 2D array probe in which a plurality of piezoelectric transducers are arrayed in a two-dimensional matrix. The ultrasound probe 1 may be, for example, a mechanical 4D probe configured to execute ultrasound scanning by mechanically prompting piezoelectric transducers aligned in a column in a direction orthogonal to the direction of the alignment.

An ultrasound wave transmitted from the ultrasound probe 1 to the subject is repeatedly reflected by discontinuous surfaces of acoustic impedances at a tissue in the body of the subject, and is received by the piezoelectric transducers as reflected-wave signals (echo signals). An amplitude of the received reflected-wave signal depends on a difference between the acoustic impedances on the discontinuous surfaces at which the ultrasound waves are reflected. It is to be noted that, if a transmitted ultrasound pulse is reflected at a surface of a moving blood flow, a cardiac wall, or the like, the reflected-wave signal is, due to the Doppler effect, subject to a frequency shift, depending on a velocity component of the moving members with respect to the ultrasound wave transmission direction. The ultrasound probe 1 receives a reflected-wave signal from the subject.

For the medical tool D, the ultrasound probe 1 is configured to execute a 2D mode of executing non-3D scanning in which at least one scanning surface is formed, thereby receiving a two-dimensional reflected-wave signal. Specifically, in the 2D mode, from the perspective of frame and resolution, the ultrasound probe 1 is configured, for example, to execute scanning in which two scanning surfaces (a Bi-Plane and an xPlane) orthogonal to one another are simultaneously or repeatedly formed. A 2D mode in which two orthogonal tomographic images (a double orthogonal cross-sectional image) are generated by executing such scanning may be referred to as a “biplane mode”. It is to be noted that the configuration is not limited thereto, and the ultrasound probe 1 may be configured to execute scanning in which three or more scanning surfaces are simultaneously or repeatedly formed. Alternatively, the ultrasound probe 1 may be configured, for example, to execute, in the 2D mode, 2D scanning in which a plurality of scanning surfaces are repeatedly formed. In this case, the apparatus main body 2 generates a single 2D cross-sectional image. In the 2D scanning, the ultrasound probe 1 is configured to change a scanning surface angle of the ultrasound probe 1 by mechanically rotating the plurality of piezoelectric transducers. The ultrasound probe 1 is configured to receive a two-dimensional reflected-wave signal according to the scanning surface angle. The ultrasound probe 1 is also configured to receive a three-dimensional reflected-wave signal by executing a 3D mode of executing 3D scanning for the medical tool D. Specifically, in the 3D scanning, the ultrasound probe 1 is configured to receive a three-dimensional reflected-wave signal by mechanically rotating the plurality of piezoelectric transducers. It is to be noted that the non-3D scanning does not include 4D scanning by a mechanical 4D probe, etc. The ultrasound probe 1 is configured to be suitably switchable from one of the 2D mode and the 3D mode to the other mode. It is to be noted that the 3D scanning is an example of first scanning. It is also to be noted that the non-3D scanning is an example of second scanning.

The input interface 11 is a user interface of various types arranged on the handle part. Examples of the input interface 11 include a button to be pressed down for offset processing or in the event of ultrasound image freezing, for example. Examples of the input interface 11 further include a mode switch button configured to receive, as an input, an instruction to switch from one of the 2D and 3D modes to the other mode.

Examples of the input interface 11 further include an angle adjustment button to be pressed down for adjustment of the scanning surface angle. In the 2D mode, the operator presses down the angle adjustment button for adjustment of the scanning surface angle to observe a target to be imaged such as the tip end part of the medical tool D and a portion other than the tip end part such as the heart valve from various angles.

Examples of the input interface 11 further include an execution instruction input button configured to receive, as an input, an execution instruction to execute 3D scanning in the background during displaying of a two-dimensional ultrasound image. The input interface 11 is configured to transmit the received execution instruction to the processing circuitry 25. It is to be noted that the execution instruction input button is an optionally additional feature, and may be omitted. The input interface 11 is an example of a first input unit.

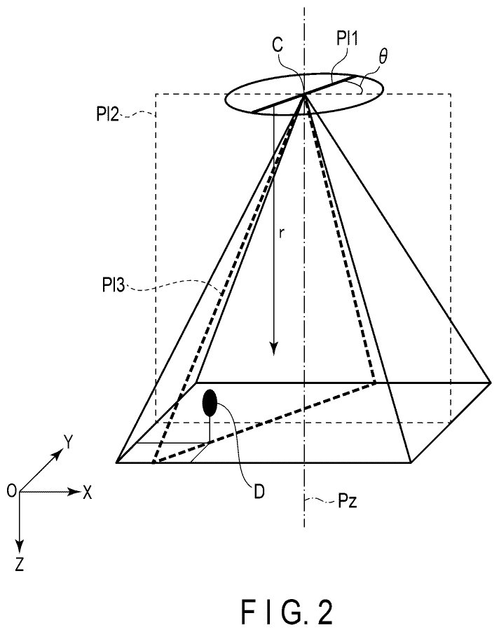

Now, a position of the ultrasound probe 1 in a three-dimensional space, a posture of the ultrasound probe 1 at that position, an angle of a scanning surface of the ultrasound probe 1, and the like will be described with reference to FIG. 2. The ultrasound probe 1 is configured to generate an ultrasound wave from a transducer-aligned surface Pl1 on which a plurality of piezoelectric transducers are one-dimensionally aligned, as shown in FIG. 2. It is assumed that the ultrasound probe 1 in a three-dimensional orthogonal coordinate system (x, y, z) in which a predetermined position is defined as an origin O is positioned at a center C (cx, cy, cz) of the transducer-aligned surface Pl1. Herein, the posture of the ultrasound probe 1 is defined by a normal vector r perpendicular to the transducer-aligned surface Pl1 and indicating a direction of transmission of ultrasound waves. Specifically, the posture of the ultrasound probe 1 is represented by components (rx, ry, rz) of the normal vector r. It is assumed that the three-dimensional orthogonal coordinate system is, for example, a right-handed coordinate system in which a direction opposite to the direction of gravity is defined as a positive direction on a z-axis. It is assumed that an x-axis is parallel to the longitudinal direction of a couch (not illustrated) on which the subject is placed. It is assumed that a y-axis is parallel to a widthwise direction of the couch. The transducer-aligned surface Pl1 is configured to rotate around a straight line passing through the center C and orthogonal to the transducer-aligned surface Pl1, defined as a rotation axis Pz, as shown in FIG. 2. In FIG. 2, a plane Pl2 represents a plane corresponding to a scanning surface in the 2D mode in which the scanning surface angle is an initial angle that has not yet been changed. In FIG. 2, a plane Pl3 represents a scanning surface in the 2D mode in which the scanning surface is at an angle θ relative to the initial angle. The scanning surface includes a medical tool D, as shown in FIG. 2. Hereinafter, the scanning surface including the medical tool D will be referred to as a “reference cross-section”. Also, a set of information items including a position, a posture angle, and a scanning surface angle of the ultrasound probe 1 (cx, cy, cz, rx, ry, rz, θ) will be referred to as “position information”. It is to be noted that the term “position information” may be suitably replaced with, for example, “positional parameters”.

The apparatus main body 2 is configured to generate an ultrasound image based on a reflected-wave signal received by the ultrasound probe 1. The apparatus main body 2 is configured to generate a two-dimensional ultrasound image based on a two-dimensional reflected-wave signal received mainly in the 2D mode. The apparatus main body 2 is configured, for example, to generate a double orthogonal cross-sectional image by executing the biplane mode. The apparatus main body 2 may be further configured to generate a single 2D cross-sectional image by executing 2D scanning. Also, the apparatus main body 2 is configured to generate a three-dimensional ultrasound image based on a three-dimensional reflected-wave signal received during the 3D scanning. The apparatus main body 2 is a computer including transmission circuitry 21, reception circuitry 22, transmission and reception control circuitry 23, generating circuitry 24, processing circuitry 25, a memory 26, an input interface 27, a communication interface 28, and a display 29.

The transmission circuitry 21 is configured to transmit a beam-shaped ultrasound wave via the ultrasound probe 1 in accordance with control of the transmission and reception control circuitry 23. Specifically, the transmission circuitry 21 is configured to supply each of the drive signals by applying thereto a delay time for giving a transmission directivity to transmit a beam-shaped ultrasound wave polarized at a given transmission beam angle. The transmission circuitry 21 is configured to repeatedly transmit an ultrasound wave by changing the transmission beam angle.

The reception circuitry 22 is configured to subject the reflected-wave signals received by the ultrasound probe 1 to various processes, and generates reception signals, in accordance with control of the transmission and reception control circuitry 23. Specifically, the reception circuitry 22 is realized by, for example, a preamplifier (a preamplifier group), an A/D converter, a demodulator, and a beam former (a reception delay adder circuit), etc. The preamplifier is configured to perform a gain correction process by amplifying the reflected-wave signals for each channel. The A/D converter is configured to convert the gain-corrected reflected-wave signals into digital signals. The demodulator is configured to demodulate the digital signals. The beam former is configured, for example, to apply a delay time required for determining a reception directivity to each of the demodulated digital signals, and summing the digital signals to which the delay time has been applied. As a result of the summing by the beam former, a reception signal with an emphasis on reflection components from a direction corresponding to the reception directivity are generated.

The transmission and reception control circuitry 23 is configured, under the control of the processing circuitry 25, to control the transmission circuitry 21 and the reception circuitry 22 in a synchronized manner to execute the non-3D scanning or 3D scanning of the target to be imaged via the ultrasound probe 1.

The generating circuitry 24 is configured to perform a B-mode process, a color Doppler process, or the like on the reception signal transmitted from the reception circuitry 22. In the B-mode process, the generating circuitry 24 is configured to perform a logarithmic amplification, an envelope detection process, a logarithmic compression, etc. on the reception signal, thereby generating, for each sample point, B-mode information in which the intensity of the signal is expressed by a degree of brightness. The generating circuitry 24 is further configured to generate, based on the B-mode information, a two-dimensional or three-dimensional B-mode image in which the intensity of the signal is expressed by a brightness value. In the Doppler process, the generating circuitry 24 is configured to perform a frequency analysis of the reception signal, and to estimate, for each sample point, Doppler information containing a velocity, a dispersion, a power, etc. of moving bodies such as blood and tissues. The generating circuitry 24 is further configured to generate a two-dimensional or three-dimensional Doppler image in which the velocity, the dispersion, the power, etc. of the moving bodies such as blood and tissues are expressed by color values. If there is no distinction between the B-mode image and the Doppler image, they will be collectively referred to as an “ultrasound image”. The ultrasound image is stored in the memory 26. The generating circuitry 24 can be realized by a given processor.

The generating circuitry 24 is configured to generate a two-dimensional ultrasound image of a subject into which the medical tool D has been inserted by executing, via the ultrasound probe 1, the non-3D scanning of the subject. The generating circuitry 24 is configured, for example, to generate a two-dimensional ultrasound image such as a mid-esphogeal five-chamber view, a mid-esphogeal two-chamber view, or the like by executing the non-3D scanning. If the position information of the ultrasound probe 1 is position information with which a two-dimensional ultrasound image containing the medical tool D can be generated, the generating circuitry 24 is configured to generate a two-dimensional ultrasound image containing the medical tool D. Hereinafter, the position information will be referred to as “first position information”. It is to be noted that the generating circuitry 24 is configured to generate a two-dimensional ultrasound image containing the medical tool D by executing the non-3D scanning via the ultrasound probe 1 whose position information has been updated to the first position information. Specifically, the generating circuitry 24 is configured to generate a two-dimensional ultrasound image containing the medical tool D if the scanning surface in the non-3D scanning corresponds to the reference cross-section. Also, the generating circuitry 24 is configured to generate a two-dimensional ultrasound image not containing the medical tool D if the position information of the ultrasound probe 1 is other position information different from the first position information. Hereinafter, such other position information will be referred to as “second position information”. Specifically, the generating circuitry 24 is configured to generate a two-dimensional ultrasound image not containing the medical tool D if the scanning surface in the non-3D scanning does not correspond to the reference cross-section.

Also, the generating circuitry 24 is configured to generate a three-dimensional ultrasound image containing the medical tool D by executing, in the background, the 3D scanning involving the non-3D scanning during displaying of the two-dimensional ultrasound image. The generating circuitry 24 is configured, for example, to generate an ultrasound image in units of frames or volumes in accordance with a preset non-3D-scanning frame rate or 3D-scanning volume rate. The generating circuitry 24 is configured to generate a two-dimensional ultrasound image by executing mainly the non-3D scanning. Also, the generating circuitry 24 is configured to generate a three-dimensional ultrasound image by executing the 3D scanning based on an execution instruction. The 3D scanning is executed for a single volume, for example. Accordingly, the generating circuitry 24 is configured to generate a single three-dimensional ultrasound image containing the medical tool D. It is to be noted that the generating circuitry 24 may be configured to generate two or more three-dimensional ultrasound images by executing the 3D scanning for two or more volumes. After generating the three-dimensional ultrasound image, the generating circuitry 24 is configured to generate a two-dimensional ultrasound image again by executing the non-3D scanning. It is to be noted that “executing the 3D scanning in the background” means that, the 3D scanning is executed for a short period of time (for a single volume) during the non-3D scanning by switching from the non-3D scanning, and then the non-3D scanning is executed again. The generating circuitry 24 is an example of a first generating unit or a second generating unit. The two-dimensional ultrasound image is an example of a first ultrasound image. The three-dimensional ultrasound image is an example of a second ultrasound image.

The processing circuitry 25 includes a processor such as a central processing unit (CPU) configured to manage the ultrasound diagnosis apparatus 100. The processing circuitry 25 is configured to execute ultrasound diagnosis programs stored in the memory 26, thereby executing functions corresponding to the respective programs. The processing circuitry 25 is configured, for example, to realize a scanning control function 251, an estimating function 252, an updating function 253, and a display controlling function 254. The functions 251 to 254 are not necessarily realized by a single unit of processing circuitry 25. The processing circuitry 25 may be configured by a combination of a plurality of independent processors configured to execute control programs into which an ultrasound diagnosis program is divided, thereby realizing the functions 251 to 254. The functions 251 to 254 may be implemented as modules configuring the control programs, or may be implemented as individual hardware units. The processing circuitry 25 may include generating circuitry 24. The same applies to the functions of the processing circuitry 25 to be discussed below in the second and subsequent embodiments.

Through realizing the scanning control function 251, the processing circuitry 25 is configured to execute the non-3D scanning of a target to be imaged in the subject via the ultrasound probe 1. Also, the processing circuitry 25 is configured to execute the 3D scanning involving the non-3D scanning during displaying of the two-dimensional ultrasound image. Specifically, the processing circuitry 25 is configured to control the transmission and reception control circuitry 23 to execute the non-3D scanning or 3D scanning. The processing circuitry 25 is configured to execute the 3D scanning for a single volume based on an execution instruction from the input interface 11. After executing the 3D scanning, the processing circuitry 25 is configured to execute the non-3D scanning via the ultrasound probe 1. It is to be noted that the scanning control function 251 is an example of the first generating unit or the second generating unit. In the present embodiment, an example has been described in which the second generating unit is configured to generate a three-dimensional ultrasound image containing the medical tool D by executing the 3D scanning in the background during displaying of a two-dimensional ultrasound image; however, the configuration is not limited thereto. The second generating unit may be configured to generate the three-dimensional ultrasound image even in a state in which the non-3D scanning is not being executed and a two-dimensional ultrasound image is not being displayed. In other words, the second generating unit is not necessarily configured to execute the 3D scanning in the background.

Through realizing the estimating function 252, the processing circuitry 25 is configured to estimate first position information based on a three-dimensional ultrasound image. The processing circuitry 25 is configured, for example, to estimate the first position information based on a three-dimensional ultrasound image generated in a state in which the position information of the ultrasound probe 1 is the second position information. Specifically, the processing circuitry 25 is configured to estimate, as the first position information, a position, a posture, and a scanning surface angle of the ultrasound probe 1 with which a two-dimensional ultrasound image containing the medical tool D can be generated. The processing circuitry 25 is configured, for example, to estimate a position and a posture included in the second position information as a position and a posture included in the first position information. On the other hand, the processing circuitry 25 is configured, for example, to estimate an angle different from the scanning surface angle included in the second position information as a scanning surface angle included in the first position information. Specifically, it is assumed, for example, that the second position information is (cx, cy, cz, rx, ry, rz, 160°). At this time, the processing circuitry 25 is configured, for example, to estimate (cx, cy, cz, rx, ry, rz, 90°) as the first position information. In the above-described example, the position and posture (cx to rz) included in the second position information are identical to the position and posture (cx to rz) included in the first position information, and the scanning surface angle (160°) included in the second position information differs from the scanning surface angle (90°) included in the first position information. Also, the processing circuitry 25 may be configured to estimate the first position information by inputting the generated three-dimensional ultrasound image to a trained model Md10, to be discussed below. It is to be noted that the estimating function 252 is an example of an estimating unit.

Through realizing the updating function 253, the processing circuitry 25 is configured to update the position information of the ultrasound probe 1 from the second position information to the first position information. Specifically, the processing circuitry 25 is configured to update the position information of the ultrasound probe 1 by changing the scanning surface angle from the scanning surface angle included in the second position information to the scanning surface angle included in the first position information. The processing circuitry 25 is configured, for example, to control the motor. The motor is configured to generate power for rotating the piezoelectric transducers in accordance with control of the processing circuitry 25. Upon receiving the power from the motor, the piezoelectric transducers rotate around the rotation axis. Through the rotating of the piezoelectric transducers, the scanning surface angle is changed. In the above-described example, the processing circuitry 25 is configured to rotate the piezoelectric transducers to change the scanning surface angle (160°) included in the second position information to the scanning surface angle (90°) included in the first position information. It is to be noted that the updating function 253 is an optionally additional feature, and may be omitted. That is, after executing the process of estimating the first position information, the processing circuitry 25 may be configured to perform a process of automatically updating the position information, or to not perform such a process. It is to be noted that the updating function 253 is an example of an updating unit.

Through realizing the display controlling function 254, the processing circuitry 25 is configured to cause the display 29 to display a two-dimensional or three-dimensional ultrasound image. The processing circuitry 25 is configured to cause the display 29 to display a two-dimensional ultrasound image containing the medical tool D, which has been generated by executing the non-3D scanning with the ultrasound probe 1 whose position information has been updated to the first position information. Specifically, the processing circuitry 25 is configured to cause the display 29 to display a two-dimensional ultrasound image containing the medical tool D, which has been automatically generated after the updating of the position information. The processing circuitry 25 is configured, for example, to cause the display 29 to display a three-dimensional ultrasound image only in the case where a display instruction has been received from the operator. It is to be noted that the processing circuitry 25 may be configured to cause the display 29 to display a three-dimensional ultrasound image even if a display instruction has not been received from the operator. Also, the displaying of the two-dimensional ultrasound image after the updating of the position information is an optionally additional feature, and may be omitted. That is, after the updating of the position information, the processing circuitry 25 may be configured to perform a process of automatically displaying the two-dimensional ultrasound image, or to not perform such a process. It is to be noted that the display controlling function 254 is an example of a display controlling unit.

The memory 26 is a memory such as a ROM and a RAM configured to store various types of information, a hard disk drive (HDD), a solid-state drive (SSD), an integrated-circuit memory, or the like. The memory 26 may be a drive device, etc. configured to read and write a variety of information to and from a portable storage medium such as a CD-ROM drive, a DVD drive, a flash memory, or the like. The memory 26 is configured, for example, to store an ultrasound diagnosis program, etc. of the ultrasound diagnosis apparatus 100. Such a program may be stored in advance in, for example, the memory 26. Also, such a program may be, for example, stored and distributed in a non-transitory computer-readable storage medium, read from the non-transitory computer-readable storage medium, and installed in the memory 26. The memory 26 is configured to store, for example, a single trained model Md10. The memory 26 may be configured to store a trained model Md10 in advance at the time of a delivery of the ultrasound diagnosis apparatus 100. Alternatively, the memory 26 may be configured to store a trained model Md10 acquired from a server device (not illustrated) after a delivery of the ultrasound diagnosis apparatus 100.

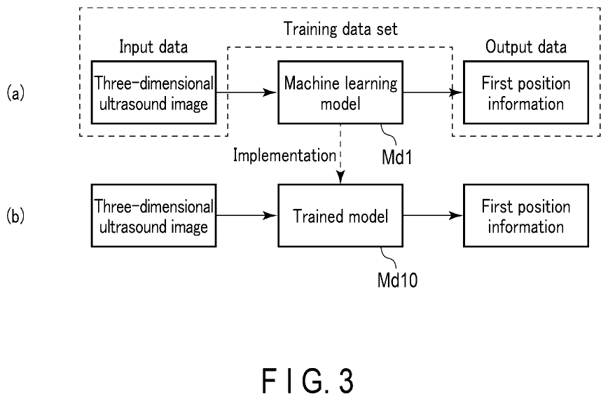

The trained model Md10 is a machine-based trained model obtained by machine-based training of a machine learning model Md1 on training data in accordance with a model training program. The machine learning model Md1 is, for example, a deep neural network (DNN) employing a convolutional neural network (CNN). An example of training of the machine learning model Md1 according to the present embodiment is shown in FIG. 3(a). A previously acquired three-dimensional ultrasound image for training, containing the medical tool D, is used as input data constituting a data set for training of the machine learning model Md1. Specifically, a previously acquired three-dimensional ultrasound image for training containing a clip is used as the input data. Also, first position information of the ultrasound probe 1 with which a two-dimensional ultrasound image for training containing the medical tool D can be generated is used as output data constituting the data set for training of the machine learning model Md1. Specifically, first position information of the ultrasound probe 1 with which a two-dimensional ultrasound image containing the clip can be generated is used as the output data. It is to be noted that the configuration is not limited thereto, and a previously acquired three-dimensional ultrasound image for training containing another medical tool D such as an artificial valve, a catheter, a guide wire, or the like may be used as the input data. Moreover, first position information of the ultrasound probe 1 with which a two-dimensional ultrasound image for training containing such another medical tool D can be generated may be used as the output data. In training, machine-based training related to a single type of medical tool is performed on a machine learning model Md1. It is to be noted that the configuration is not limited thereto, and, in training, machine-based training related to a plurality of types of medical tools may be performed on a machine learning model Md1.

Also, the trained model Md10 is a trained machine learning model Md1 that has been implemented as shown for example in FIG. 3(b). Upon taking, as an input, a three-dimensional ultrasound image containing the medical tool D, which has been generated by executing 3D scanning during examination, the trained model Md10 is configured to output first position information of the ultrasound probe 1 with which a two-dimensional ultrasound image containing the medical tool D can be generated. Specifically, upon taking, as an input, a three-dimensional ultrasound image containing a clip, which has been generated through execution of 3D scanning during examination, for example, the trained model Md10 is configured to output first position information of the ultrasound probe 1 with which a two-dimensional ultrasound image containing the clip can be generated. Also, upon taking, as an input, a three-dimensional medical image containing another medical tool D such as a catheter or a guide wire, the trained model Md10 is configured to output first position information of the ultrasound probe 1 with which the two-dimensional ultrasound image containing the medical tool D can be generated. It is to be noted that the trained model Md10 may be configured to output only a scanning surface angle of the ultrasound probe 1 at which a two-dimensional ultrasound image containing the medical tool D can be generated.

The input interface 27 of the apparatus main body 2 is, for example, a user interface of various types on a touch panel, an operation panel, or the like. The input interface 27 allows the operator to input various operations and instructions related to the ultrasound diagnosis apparatus 100. The above-described buttons may be provided on the input interface 27 of the apparatus main body 2.

The communication interface 28 is configured to perform data communications with a picture archiving and communication system (PACS) server, a hospital information system (HIS) server, a modality worklist management (MWM) server, or the like via a local area network (LAN), etc.

The display 29 is configured to display various types of information in accordance with an instruction from the processing circuitry 25. As the display 29, for example, a CRT display, a liquid crystal display (LCD), a cathode-ray tube (CRT) display, an organic electroluminescent display (OELD), a plasma display, or any other given displays may be suitably used. It is to be noted that a projector may be provided as the display 29. The display 29 is configured, for example, to display a two-dimensional ultrasound image generated through execution of the non-3D scanning. The display 29 is further configured to display a two-dimensional ultrasound image containing the medical tool D, which has been generated through execution of non-3D scanning with the ultrasound probe 1 whose position information has been updated to the first position information.

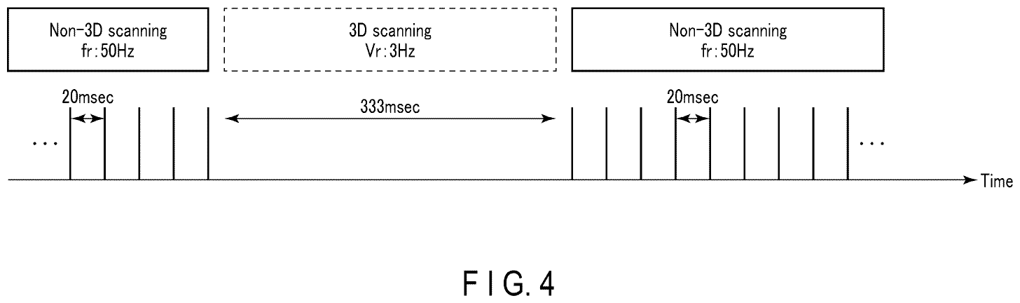

Next, an example of an operation of the ultrasound diagnosis apparatus 100 with the above-described configuration will be described with reference to FIGS. 4 to 8. In the following, an operation of the ultrasound diagnosis apparatus 100 during a procedure of percutaneous mitral valve clipping, which is a type of catheter intervention, will be described.

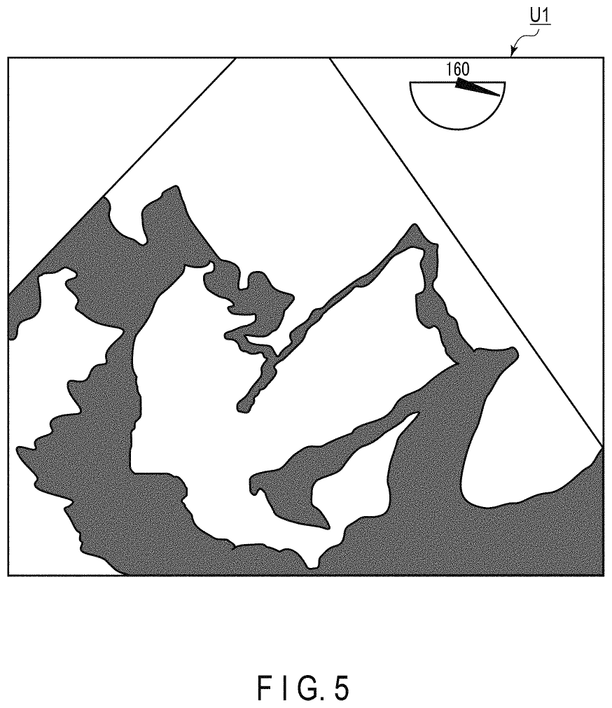

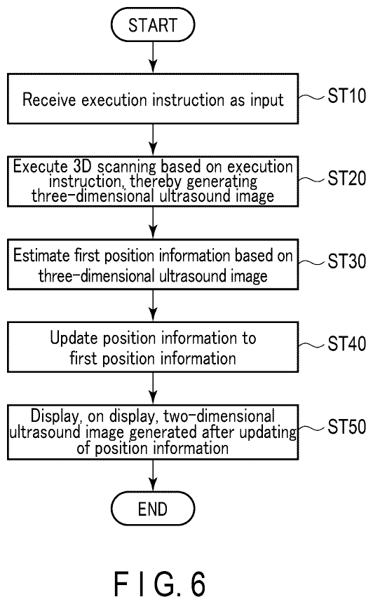

First, the ultrasound diagnosis apparatus 100 executes, during the procedure, a 2D mode on the heart of the subject into which a clip has been inserted, and generates and displays a two-dimensional ultrasound image containing the tip end part of the clip. At this time, a frame rate fr in the non-3D scanning is set to, for example, 50 Hz, as shown in the left part of FIG. 4. In this case, a scanning time per frame is 20 milliseconds. If, for example, the operator has lost sight of the tip end part of the clip, or is to observe a portion other than the tip end part such as the heart valve, the ultrasound diagnosis apparatus 100 causes the display 29 to display a two-dimensional ultrasound image U1 (a mid-esphogeal five-chamber view) not containing the tip end part, as shown in FIG. 5. It is assumed that, in FIG. 5, a clip is not contained by the two-dimensional ultrasound image U1. It is assumed, at this time, that the position information of the ultrasound probe 1 is the second position information (cx, cy, cz, rx, ry, rz, 160°). After the operator has confirmed that the two-dimensional ultrasound image U1 does not contain the medical tool D, step ST10 is performed, as shown in FIG. 6.

At step ST10, the input interface 11 provided on the ultrasound probe 1 receives, as an input, a 3D scanning execution instruction through an input operation by an operator who has confirmed that the two-dimensional ultrasound image U1 being displayed does not contain the medical tool D. The input interface 11 is configured to transmit the received execution instruction to the processing circuitry 25.

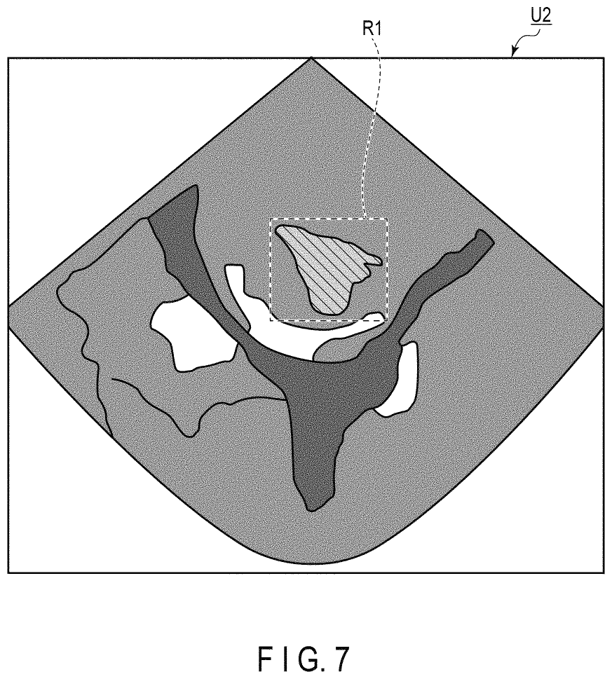

After step ST10, the processing circuitry 25 executes, at step ST20, 3D scanning for a single volume based on the execution instruction from the input interface 11. At this time, the volume rate Vr is set to, for example, 3 Hz, as shown at the center of FIG. 4. In this case, a scanning time per volume data is approximately 333 milliseconds. Through executing the 3D scanning for a single volume, the generating circuitry 24 generates a three-dimensional ultrasound image U2 containing a clip, as shown in FIG. 7. After executing the 3D scanning, the processing circuitry 25 executes non-3D scanning again. It is to be noted that the three-dimensional ultrasound image U2 contains a clip, as shown in a region R1 in FIG. 7.

After step ST20, the processing circuitry 25 inputs, at step ST30, the three-dimensional ultrasound image U2 containing the clip to a trained model Md10. This allows the processing circuitry 25 to estimate first position information (90°), which is position information of the ultrasound probe 1 with which a two-dimensional ultrasound image containing the clip can be generated.

After step ST30, the processing circuitry 25 changes, at step ST40, a scanning surface angle from a scanning surface angle (160°) included in the second position information to the scanning surface angle (90°) included in the first position information by rotating a plurality of piezoelectric transducers.

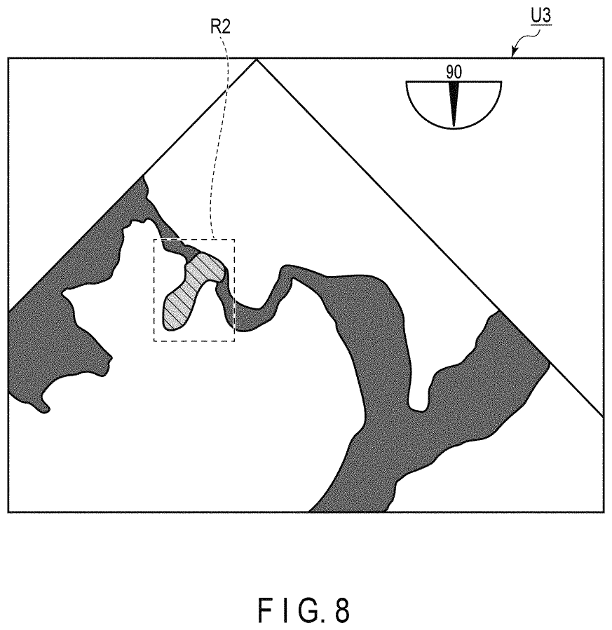

After step ST40, the generating circuitry 24 generates, at step ST50, a two-dimensional ultrasound image U3 (a mid-esphogeal two-chamber view) at a scanning surface angle of 90° through execution of the non-3D scanning, as shown in FIG. 8. The processing circuitry 25 causes the display 29 to display the generated two-dimensional ultrasound image U3. It is to be noted that the two-dimensional ultrasound image U3 contains the clip, as shown in a region R2 in FIG. 8. At steps ST10 to ST50 described above, the processing circuitry 25 switches from the non-3D scanning to execute 3D scanning during image displaying in the non-3D scanning, and outputs the estimated angle (90°) of the first scanning surface to the display 29. After step ST50, the processing ends.

It is to be noted that an example of the operation in a percutaneous mitral valve clipping procedure has been described above; however, the configuration is not limited thereto. The ultrasound diagnosis apparatus 100 may be configured, for example, to operate during a transcatheter aortic valve implantation procedure. For such an operation, the “clip” in the description that precedes should be replaced with an “artificial valve”. In a catheter intervention, there is a case where, for example, the operator who is observing a tip end part of a catheter has lost sight of the tip end part of the catheter due to moving the tip end part of the catheter. For the operation of the ultrasound diagnosis apparatus 100 in such a case, the “clip” in the description that precedes should be replaced with a “catheter”.

According to the first embodiment described above, the ultrasound diagnosis apparatus 100 includes generating circuitry 24 (a first generating unit or a second generating unit) and processing circuitry 25 configured to realize a scanning control function 251 (a first generating unit or a second generating unit), and an estimating function 252 (an estimating unit). The processing circuitry 25 is configured to execute, via the ultrasound probe 1, non-3D scanning (first scanning) of a subject into which a medical tool D has been inserted. The generating circuitry 24 is configured to generate a two-dimensional ultrasound image (a first ultrasound image) of the subject by executing non-3D scanning. The processing circuitry 25 is configured to execute 3D scanning (second scanning) involving the non-3D scanning. This allows the generating circuitry 24 to generate a three-dimensional ultrasound image (a second ultrasound image) containing the medical tool D. The processing circuitry 25 is configured to estimate, based on a three-dimensional ultrasound image, first position information of the ultrasound probe 1 with which a two-dimensional ultrasound image containing the medical tool D can be generated. The processing circuitry 25 switches from the non-3D scanning to execute 3D scanning during image displaying in the non-3D scanning, and outputs the estimated angle of the first scanning surface. As described above, the ultrasound diagnosis apparatus 100 is configured to generate a three-dimensional ultrasound image by executing 3D scanning, and to estimate first position information based on the three-dimensional ultrasound image. By thus performing a series of processing from execution of the second scanning to estimation of the first position information, the ultrasound diagnosis apparatus 100 can support the operator's operation of searching for the medical tool D on the ultrasound image.

That is, the ultrasound diagnosis apparatus 100 can support the operator's operation of searching for a medical tool D such as a clip placed on the mitral valve, an artificial valve replaced from the aortic valve, etc. on an ultrasound image. It is thereby possible to support a catheter intervention for a structural heart disease, such as percutaneous mitral valve clipping and transcatheter aortic valve implantation.

Now, an ultrasound diagnosis apparatus according to a comparative example will be described. The ultrasound diagnosis apparatus according to the comparative example is not configured to perform the above-described series of processing. Accordingly, the ultrasound diagnosis apparatus according to the comparative example is configured to receive, as an input, a switching instruction to switch from the 2D mode to the 3D mode to allow the operator to grasp an overall image of a region including the medical tool D to observe the tip end part again. Upon receiving, as an input, the switching instruction, the ultrasound diagnosis apparatus according to the comparative example is configured to switch from the 2D mode to the 3D mode, and to cause a display to display a three-dimensional ultrasound image. Normally, such a process is not particularly problematic; however, causing a display to display a three-dimensional ultrasound image incurs an increase in the processing amount of the ultrasound diagnosis apparatus, compared to the case of executing a 2D mode, resulting in an increase in processing time and a greater load to be placed on the processor.

On the other hand, in the ultrasound diagnosis apparatus 100 according to the first embodiment, 3D scanning is executed only for a single volume in the above-described series of processing, and a process of causing the display 29 to display a three-dimensional ultrasound image is omitted. This reduces the processing amount of the ultrasound diagnosis apparatus 100, thus resulting in reduction in the processing time and reduction in the load on the processor.

According to the first embodiment, the ultrasound diagnosis apparatus 100 further includes an execution instruction input button (a first input unit) configured to receive, as an input, a 3D scanning execution instruction. The processing circuitry 25 is configured to execute 3D scanning based on the execution instruction. This allows the operator to perform the above-described series of processing with only a button input to grasp position information of the medical tool D on the ultrasound image, thus making it possible to support the operator's operation of searching for the medical tool D, in addition to achieving the above-described effect.

According to the first embodiment, the first scanning is non-3D scanning in which at least one scanning surface is formed. With the ultrasound diagnosis apparatus 100, it is thereby possible to generate a two-dimensional ultrasound image such as a double orthogonal cross-sectional image and a 2D cross-sectional image by executing scanning in which two orthogonal scanning surfaces are formed and 2D scanning in which a single scanning surface is formed, in addition to achieving the above-described effect.

Also, according to the first embodiment, the second scanning is 3D scanning. It is thereby possible, with the ultrasound diagnosis apparatus 100, to generate a three-dimensional ultrasound image by executing the 3D scanning, in addition to achieving the above-described effect.

Also, according to the first embodiment, the processing circuitry 25 is further equipped with an updating function 253 of updating position information of the ultrasound probe 1 from second position information to first position information. It is thereby possible to automatically update the position information of the ultrasound probe 1 to position information of the ultrasound probe 1 with which a two-dimensional ultrasound image containing the medical tool D can be generated, in addition to achieving the above-described effect.

Also, according to the first embodiment, the first position information includes a scanning surface angle of the ultrasound probe 1 at which the two-dimensional ultrasound image can be generated. The processing circuitry 25 is configured to update the position information of the ultrasound probe 1 by changing the scanning surface angle from the scanning surface angle included in the second position information to the scanning surface angle included in the first position information. It is thereby possible to update the position information of the ultrasound probe 1 to position information of the ultrasound probe 1 with which a two-dimensional ultrasound image containing the medical tool D can be generated by changing the scanning surface angle, in addition to achieving the above-described effect.

It is to be noted that, according to the first embodiment, the generating circuitry 24 is configured to generate a two-dimensional ultrasound image containing the medical tool D by executing non-3D scanning via the ultrasound probe 1 whose position information has been updated to first position information. It is thereby possible to automatically generate a two-dimensional ultrasound image containing the medical tool D, in addition to achieving the above-described effect.

Moreover, according to the first embodiment, the processing circuitry 25 further includes a display controlling function 254 (a display controlling unit) of causing the display 29 to display a two-dimensional ultrasound image containing the medical tool D, which has been generated after updating of the position information. It is thereby possible to automatically cause the display 29 to display a two-dimensional ultrasound image containing the medical tool D, in addition to achieving the above-described effect.

Furthermore, according to the first embodiment, the processing circuitry 25 is configured to input, based on a three-dimensional ultrasound image for training containing a medical tool D, a three-dimensional ultrasound image containing the medical tool D, which has been generated by executing the 3D scanning during examination, to a trained model Md10 configured to output first position information of the ultrasound probe 1, with which a two-dimensional ultrasound image for training containing the medical tool D can be generated. The processing circuitry 25 is configured to estimate, based on the output from the trained model Md10, the first position information of the ultrasound probe 1 with which the two-dimensional ultrasound image containing the medical tool D can be generated. It is thereby possible to estimate first position information using the trained model Md10, in addition to achieving the above-described effect.

In addition, according to the first embodiment, the medical tool D includes a first medical tool or a second medical tool. The processing circuitry 25 is configured to estimate first position information of the ultrasound probe 1 with which a two-dimensional ultrasound image containing the first medical tool can be generated in the case of inputting, to the trained model Md10, a second ultrasound image containing the first medical tool. The processing circuitry 25 is configured to estimate the first position information of the ultrasound probe 1 with which a two-dimensional ultrasound image containing the second medical tool can be generated in the case of inputting, to the trained model Md10, a second ultrasound image containing the second medical tool. The processing circuitry 25 is thus configured to estimate the first position information corresponding to the medical tool no matter whether the medical tool individually contained by a three-dimensional ultrasound image input to a single trained model Md10 is the first medical tool or the second medical tool. It is thereby possible, with the above-described configuration in which a plurality of trained models need not be stored, to reduce the memory usage, in addition to achieving the above-described effect.

### Modification of First Embodiment

The first embodiment may be modified in the following manner. Each modification may be combined with another modification, or with any one of the embodiments to be described below.

According to the first embodiment, the processing circuitry 25 is configured to cause the display 29 to display a two-dimensional ultrasound image containing the medical tool D, which has been generated by executing non-3D scanning with the ultrasound probe 1 whose position information has been updated to the first position information; however, the configuration is not limited thereto. The processing circuitry 25 may be configured, for example, to cause the display 29 to display display screen data in which the above-described two-dimensional ultrasound image and a three-dimensional ultrasound image generated through execution of 3D scanning are arranged in parallel.

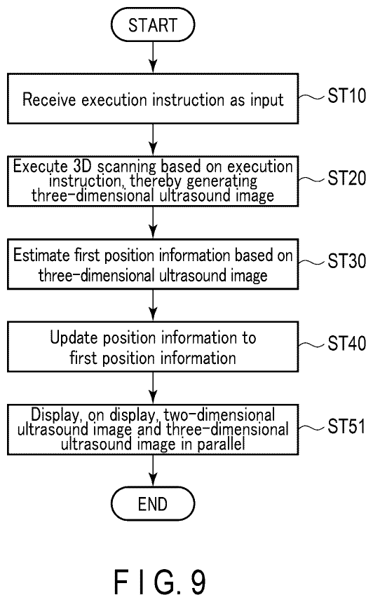

According to this modification, the ultrasound diagnosis apparatus 100 executes steps ST10 to ST40 in a manner similar to the foregoing, as shown in FIG. 9. After step ST40, the processing shifts to step ST51.

At step ST51, the ultrasound diagnosis apparatus 100 is configured to cause the display 29 to display display screen data in which a two-dimensional ultrasound image containing the medical tool D, which has been generated by the ultrasound probe 1 whose position information has been updated to the first position information, and a three-dimensional ultrasound image are arranged in parallel. After step ST51, the processing ends.

With the above-described configuration according to the modification, the operator is allowed to visually recognize the two ultrasound images on a single display screen. It is thereby possible for the operator to adjust the scanning surface angle while viewing the three-dimensional ultrasound image even if the medical tool D is not contained by the two-dimensional ultrasound image displayed after updating of the position information, in addition to achieving the effect of the first embodiment.

### Second Embodiment

In a second embodiment, which is a modification of the first embodiment, an operator is notified of first position information of an ultrasound probe 1 with which a two-dimensional ultrasound image containing a medical tool D can be generated.

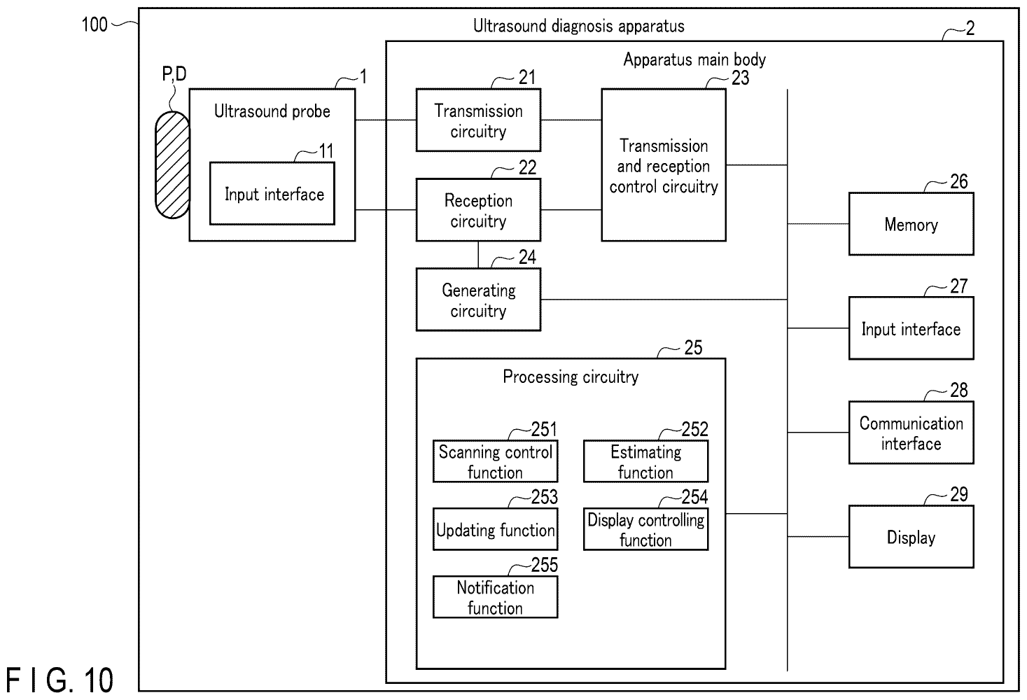

FIG. 10 shows an example of a configuration of an ultrasound diagnosis apparatus 100 according to the second embodiment. The processing circuitry 25 further includes a notification function 255, as shown in FIG. 10.

Through realizing the notification function 255, the processing circuitry 25 is configured to notify the operator of the first position information. The ultrasound diagnosis apparatus 100 is further equipped with, for example, a speaker (not illustrated). The processing circuitry 25 is configured to notify the operator of the first position information by causing the speaker to output the first position information. The configuration is not limited thereto, and the processing circuitry 25 may be configured, for example, to cause the display 29 to display the first position information, thereby notifying the operator of the first position information. At this time, the operator adjusts, for example, a scanning surface angle based on the first position information of which the notification has been made. It is to be noted that the ultrasound diagnosis apparatus 100 may have any configuration that allows the operator to be notified of the first position information. The notification function 255 is an example of a first notification unit.

The other configurations of the ultrasound diagnosis apparatus 100 are similar to those of the first embodiment.

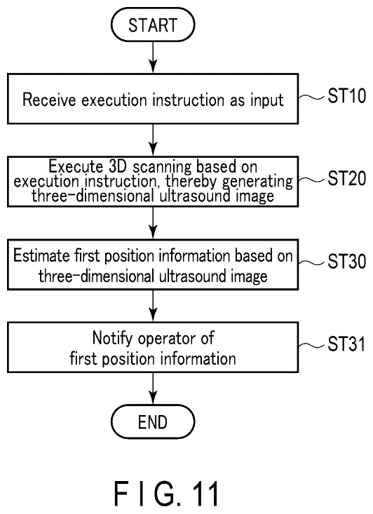

In the second embodiment, the ultrasound diagnosis apparatus 100 executes steps ST10 to ST30 in a manner similar to the foregoing, as shown in FIG. 11. After step ST30, the processing shifts to step ST31.

At ST31, the processing circuitry 25 is configured to notify the operator of the first position information by causing the speaker to output the first position information. After step ST31, the processing ends.

In the second embodiment, the processing circuitry 25 is further equipped with a notification function 255 (a first notification unit) of notifying the operator of the first position information. It is thereby possible for the operator to grasp the first position information of the ultrasound probe 1 with which a two-dimensional ultrasound image containing the medical tool D can be generated, in addition to achieving the effect of the first embodiment. That is, the operator who has grasped the position information is capable of adjusting the scanning surface angle through an operation by the operator himself/herself, regardless of automatic updating of the position information by the ultrasound diagnosis apparatus 100.

### Third Embodiment

In a third embodiment, which is a modification of the first or second embodiment, a plurality of trained models Md10 are stored, a type of a medical tool D contained by a three-dimensional ultrasound image is determined, and a trained model Md10 corresponding to the type of the medical tool D is selected.

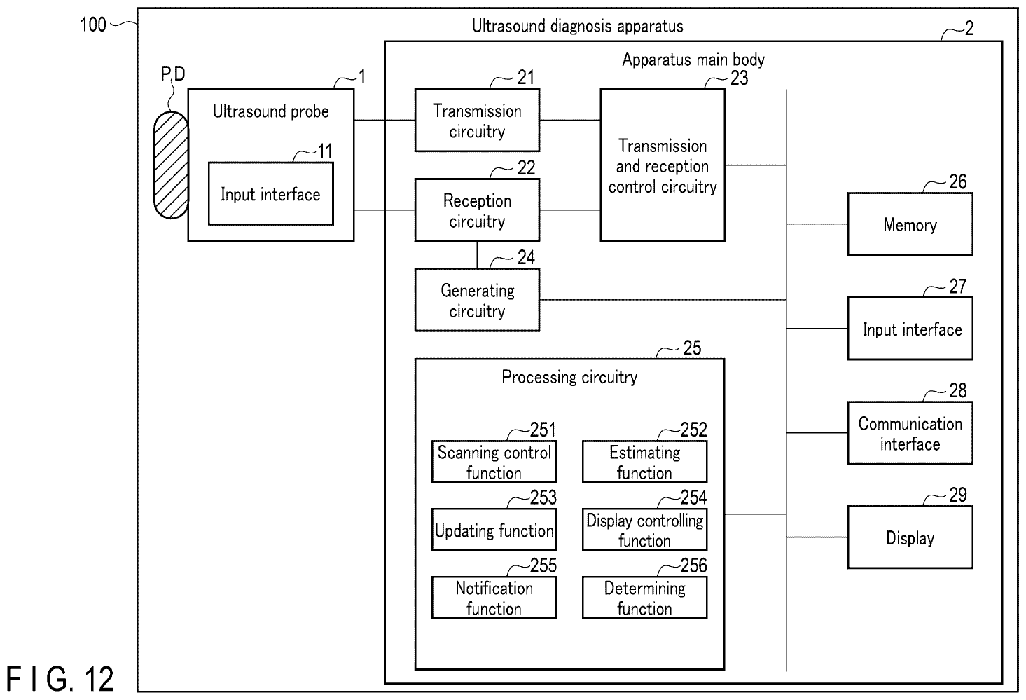

FIG. 12 shows an example of a configuration of an ultrasound diagnosis apparatus 100 according to the third embodiment. The processing circuitry 25 further includes a determining function 256, as shown in FIG. 12.

The memory 26 may be configured to store a plurality of trained models Md10, unlike the above-described configuration. The plurality of trained models Md10 include, for example, a first trained model configured to output first position information of the ultrasound probe 1 with which a two-dimensional ultrasound image containing a clip can be generated based on a three-dimensional ultrasound image containing the clip. The plurality of trained models Md10 further include a second trained model configured to output first position information of the ultrasound probe 1 with which a two-dimensional ultrasound image containing an artificial valve can be generated based on a three-dimensional ultrasound image containing the artificial valve. It is to be noted that the configuration is not limited thereto, and the memory 26 may store a trained model configured to output first position information of the ultrasound probe 1 with which a two-dimensional ultrasound image containing another medical tool D can be generated based on a three-dimensional ultrasound image containing another medical tool D such as a catheter and a guide wire. It is to be noted that the memory 26 may be configured to store three or more trained models.

Through realizing the determining function 256, the processing circuitry 25 is configured, for example, to determine whether or not the medical tool D contained by the three-dimensional ultrasound image is a clip or an artificial valve based on the three-dimensional ultrasound image. It is to be noted that the determining function 256 is an example of a determination unit.

In addition to the above-described configuration, the processing circuitry 25 is configured, through realizing the estimating function 252, to input the three-dimensional ultrasound image to the first trained model if it has been determined that the medical tool D included in the three-dimensional ultrasound image is a clip. If it has been determined that the medical tool D contained by the three-dimensional ultrasound image is an artificial valve, the processing circuitry 25 is configured to input the three-dimensional ultrasound image to the second trained model.

The other configurations of the ultrasound diagnosis apparatus 100 are similar to those of the first embodiment.

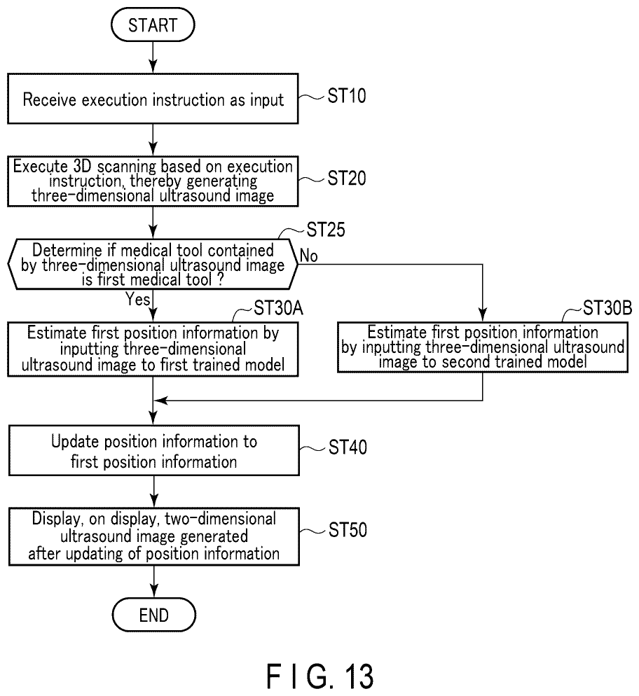

Next, an example of an operation of the ultrasound diagnosis apparatus 100 with the above-described configuration will be described with reference to the flowchart shown in FIG. 13. After the operator has confirmed, during a percutaneous mitral valve clipping procedure, that the two-dimensional ultrasound image does not contain the medical tool D, step ST10 is started, in a manner similar to the foregoing. In the present embodiment, however, the memory 26 stores both a first trained model and a second trained model.

The ultrasound diagnosis apparatus 100 executes processing at steps ST10 and ST20 in a manner similar to the foregoing. After step ST20, the processing shifts to step ST25.

After step ST20, the processing circuitry 25 determines at step ST25, based on a three-dimensional ultrasound image, whether the medical tool D contained by the three-dimensional ultrasound image is a clip (a first medical tool) or an artificial valve (a second medical tool). In this example, since the percutaneous mitral valve clipping procedure is being performed, the medical tool D contained by the three-dimensional ultrasound image is a clip. In this case, since the medical tool D contained by the three-dimensional ultrasound image is a first medical tool, the processing advances to step ST30A. It is to be noted that the medical tool D contained by the three-dimensional ultrasound image is an artificial valve if a transcatheter aortic valve implantation procedure is being performed. In this case, since the medical tool D contained by the three-dimensional ultrasound image is a not a first medical tool, the processing advances to step ST30B.

After step ST25, the processing circuitry 25 inputs, at step ST30A, a three-dimensional ultrasound image to the first trained model. This allows the processing circuitry 25 to estimate first position information of the ultrasound probe 1 with which a two-dimensional ultrasound image containing the clip can be generated.

After step ST25, the processing circuitry 25 inputs, at step ST30B, a three-dimensional ultrasound image to the second trained model. This allows the processing circuitry 25 to estimate first position information of the ultrasound probe 1 with which a two-dimensional ultrasound image containing the artificial valve can be generated.

After step ST30A or step ST30B, the ultrasound diagnosis apparatus 100 executes processing at steps ST40 and ST50 in a manner similar to the foregoing. After step ST50, the processing ends.

According to the third embodiment, the medical tool D includes a first medical tool or a second medical tool, as described above. The trained model Md10 includes a first trained model configured to output first position information of the ultrasound probe 1 with which a two-dimensional ultrasound image (a first ultrasound image) containing a first medical tool can be generated based on a three-dimensional ultrasound image (a second ultrasound image) containing the first medical tool. The trained model Md10 includes a second trained model configured to output first position information of the ultrasound probe 1 with which a two-dimensional ultrasound image containing a second medical tool can be generated based on a three-dimensional ultrasound image containing the second medical tool. In this manner, a medical tool contained by the three-dimensional ultrasound image is determined, and the medical tool D is input to the trained model Md10 corresponding to the determined medical tool D, and thereby the first position information is estimated. With the above-described configuration, it is possible to estimate first position information using a trained model Md10 specialized for a single medical tool D, thus improving the precision of estimating the first position information, in addition to achieving the effect of the first embodiment. Since the plurality of machine learning models Md1 can be trained in parallel, it is possible to reduce the time required in training.

### Modification of Third Embodiment

The third embodiment may be modified in the following manner. Each modification may be combined with another modification, or with any one of the embodiments to be described below.

In the third embodiment, the processing circuitry 25 is configured to determine a medical tool D contained by the three-dimensional ultrasound image, and to input the medical tool D to the trained model Md10 corresponding to the determined medical tool D; however, the configuration is not limited thereto. For example, the input interface 11 of the ultrasound probe 1 may be configured, in addition to the above-described configuration, to receive, as an input, a first input instruction to input a three-dimensional ultrasound image to a first trained model, or a second input instruction to input a three-dimensional ultrasound image to a second trained model. In addition to the above-described configuration, the processing circuitry 25 realizing the estimating function 252 may be configured, in addition to the above-described configuration, to input a three-dimensional ultrasound image to the first trained model based on a first input instruction, and to input a three-dimensional ultrasound image to the second trained model based on a second input instruction. Specifically, if the medical tool D is a clip, for example, the input interface 11 is configured to receive, as an input, a first input instruction by the operator. On the other hand, if the medical tool D is an artificial valve, the input interface 11 is configured to receive, as an input, a second input instruction by the operator. It is to be noted that the configuration is not limited thereto, and the input interface 11 may be configured to receive, as an input, an input instruction to input a three-dimensional ultrasound image to a trained model other than the first and second trained models. In this case, the processing circuitry 25 is configured to input, based on the input instruction, a three-dimensional ultrasound image to another trained model. The input interface 27 of the apparatus main body 2 may be further configured to receive, as an input, a first input instruction or a second input instruction. The input interface 11 is an example of a second input unit.

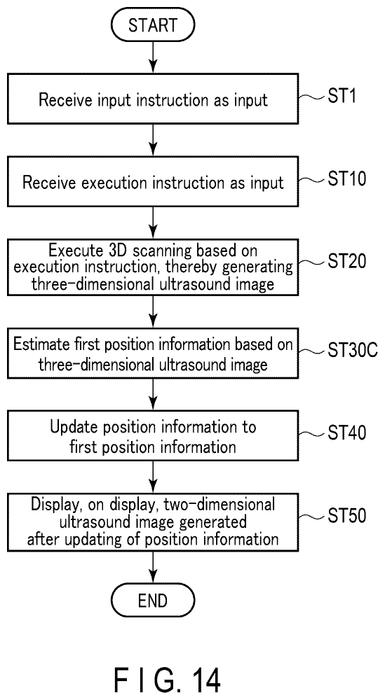

Next, an example of an operation of the ultrasound diagnosis apparatus 100 with the above-described configuration will be described with reference to the flowchart shown in FIG. 14. During a percutaneous mitral valve clipping procedure, step ST1 is started while the ultrasound diagnosis apparatus 100 is causing the display 29 to display a two-dimensional ultrasound image not containing the tip end part.

At step ST1, since the percutaneous mitral valve clipping procedure is being performed in this example, the input interface 11 receives, as an input, a first input instruction by the operator. The input interface 11 transmits the received first input instruction to the processing circuitry 25. It is to be noted that, if a transcatheter aortic valve implantation procedure is being performed, the input interface 11 receives, as an input, a second input instruction by the operator. The input instruction may be input by the operator either before or during the procedure.

After step ST1, the ultrasound diagnosis apparatus 100 executes processing at steps ST10 and ST20 in a manner similar to the foregoing.

In this example, after step ST20, the processing circuitry 25 inputs, at step ST30C, a three-dimensional ultrasound image to the first trained model based on a first input instruction. This allows the processing circuitry 25 to estimate first position information of the ultrasound probe 1 with which a two-dimensional ultrasound image containing the clip can be generated. It is to be noted that, if a transcatheter aortic valve implantation procedure is being performed, the processing circuitry 25 inputs a three-dimensional ultrasound image to the second trained model based on the second input instruction. This allows the processing circuitry 25 to estimate first position information of the ultrasound probe 1 with which a two-dimensional ultrasound image containing the artificial valve can be generated.

After step ST30C, the ultrasound diagnosis apparatus 100 executes processing at steps ST40 and ST50 in a manner similar to the foregoing. After step ST50, the processing ends.

In the above-described modification, processing of determining the medical tool D contained by the three-dimensional ultrasound image can be omitted. It is thereby possible to reduce the processing amount of the ultrasound diagnosis apparatus 100 during a period from input of the execution instruction to estimation of the first position information, thus resulting in reduction in the processing time and reduction in the load on the processor, in addition to the effect of the third embodiment.

### Fourth Embodiment

In a fourth embodiment, which is a specific example of the second or third embodiment, whether or not a medical tool D is contained by a two-dimensional ultrasound image is detected, and if the medical tool D is not contained by the two-dimensional ultrasound image, the operator is notified that it is not contained.

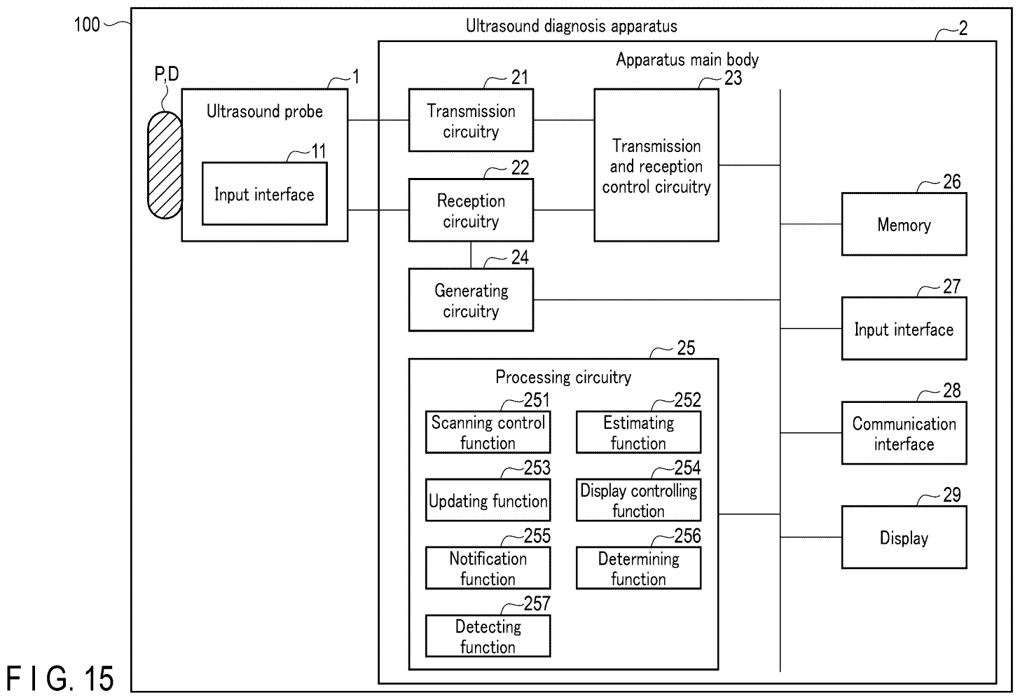

FIG. 15 shows a configuration example of an ultrasound diagnosis apparatus 100 according to the fourth embodiment. The processing circuitry 25 further includes a detecting function 257, as shown in FIG. 15.

Through realizing the detecting function 257, the processing circuitry 25 is configured to detect whether or not the medical tool D is contained by the two-dimensional ultrasound image. As a result of the detection, statuses such as “medical tool D contained” and “medical tool D not contained” may be suitably used. It is to be noted that the detecting function 257 is an example of a detection unit.

If it has been detected that the medical tool D is not contained by the two-dimensional ultrasound image, the processing circuitry 25 may be configured, in addition to the above-described configuration, to notify the operator that it is not contained, through realizing the notification function 255. The processing circuitry 25 may be configured, for example, to output the content to be notified as a speech from a speaker, or as a display on the display 29. It is to be noted that the ultrasound diagnosis apparatus 100 may have any configuration that allows the operator to be notified of the content to be notified. Also, the notification function 255 is an example of a second notification unit.

The other configurations of the ultrasound diagnosis apparatus 100 are similar to those of the second or third embodiment.

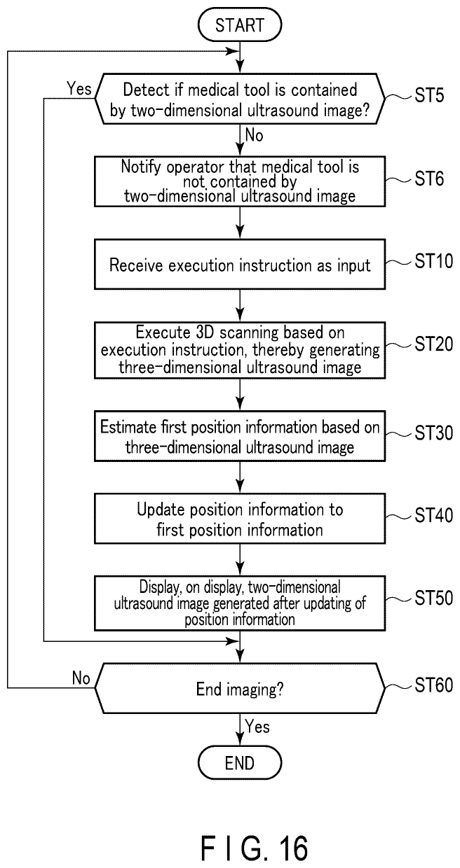

Next, an example of an operation of the ultrasound diagnosis apparatus 100 with the above-described configuration will be described with reference to the flowchart shown in FIG. 16.

First, the ultrasound diagnosis apparatus 100 executes, during a percutaneous mitral valve clipping procedure, a 2D mode on the heart of the subject into which a clip has been inserted, and generates and displays a two-dimensional ultrasound image containing a tip end part of the clip. Step ST5 is started in this state.

At step ST5, the processing circuitry 25 detects whether or not the tip end part of the clip is contained by the two-dimensional ultrasound image. If the processing circuitry 25 has detected that the tip end part of the clip is contained by the two-dimensional ultrasound image, the processing advances to step ST60. On the other hand, if the processing circuitry 25 has detected that the tip end part of the clip is not contained by the two-dimensional ultrasound image, the processing shifts to step ST6.

After step ST5, the processing circuitry 25 notifies the operator, at step ST6, that a medical tool D is not contained by the two-dimensional ultrasound image by means of a speech output from a speaker.

After step ST6, the ultrasound diagnosis apparatus 100 executes steps ST10 to ST50 in a manner similar to the foregoing.

After step ST5 or ST50, if the ultrasound imaging is not to end at step ST60 and is continued, the processing shifts to step ST5, and steps ST5 to step ST50 are repeatedly performed. On the other hand, if the ultrasound imaging is to end at step ST60, the ultrasound diagnosis apparatus 100 ends the processing.

According to the fourth embodiment, the processing circuitry 25 further includes a detecting function 257 (a detection unit) configured to detect whether or not a medical tool D is contained by a two-dimensional ultrasound image (a first ultrasound image). It is thereby possible to determine whether or not a medical tool D is contained by a two-dimensional ultrasound image even if the operator does not visually recognize the two-dimensional ultrasound image, in addition to achieving the effect of the second or third embodiment.

According to the fourth embodiment, the processing circuitry 25 further includes a notification function 255 (a second notification unit) configured, if it has been detected that the medical tool D is not contained by the two-dimensional ultrasound image, to notify the operator that it is not contained. It is thereby possible to allow the operator to grasp that the medical tool D is not contained by the two-dimensional ultrasound image even if the operator does not visually recognize the two-dimensional ultrasound image, thus prompting the operator to input an executing instruction to execute 3D scanning, in addition to achieving the above-described effect.

### Modification of Fourth Embodiment

The first embodiment may be modified in the following manner. Each modification may be combined with another modification, or with any one of the embodiments to be described below.

According to the fourth embodiment, whether or not a medical tool D is contained by a two-dimensional ultrasound image is detected, and if the medical tool D is not contained by the two-dimensional ultrasound image, the operator is notified that it is not contained; however, the configuration is not limited thereto. In addition to the above-described configuration, the generating circuitry 24 (a second generating unit) may be configured, if it has been detected that the medical tool D is not contained by the two-dimensional ultrasound image (a first ultrasound image), to execute 3D scanning (second scanning). Specifically, if the processing circuitry 25 has detected that the medical tool D is not contained by the two-dimensional ultrasound image, the ultrasound diagnosis apparatus 100 may be configured to automatically execute a series of processing from execution of 3D scanning to estimation of first position information. Also, the ultrasound diagnosis apparatus 100 may be configured to automatically update position information of the ultrasound probe 1 after a series of processing, and to execute processing of generating and displaying a two-dimensional ultrasound image.

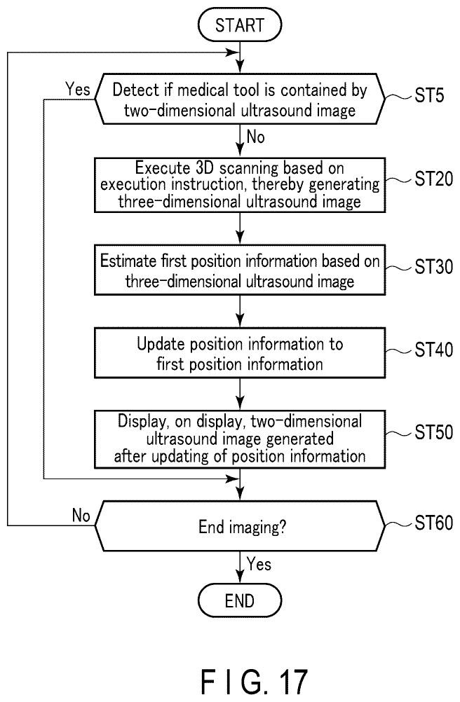

According to this modification, the ultrasound diagnosis apparatus 100 executes processing at step ST5 in a manner similar to the foregoing, as shown in FIG. 17. However, if the processing circuitry 25 has detected that the tip end part of the clip is not contained by the two-dimensional ultrasound image, the processing shifts to step ST20.

After step ST5, the ultrasound diagnosis apparatus 100 executes steps ST20 to ST60 in a manner similar to the foregoing. However, at step ST20, the processing circuitry 25 executes the 3D scanning for a single volume even if an execution instruction has not been received from the input interface 11 after detecting that the tip end part of the clip is not contained by the two-dimensional ultrasound image.

According to the configuration of the modification, after the processing circuitry 25 has detected that the medical tool D is not contained by the two-dimensional ultrasound image, a series of processing from execution of 3D scanning to estimation of the first position information is automatically executed without intervention of an operation by the operator. Also, after the series of processing, position information of the ultrasound probe 1 is updated, and processing of generating and displaying the two-dimensional ultrasound image is executed. It is thereby possible to more reliably support the operator's operation of searching for the medical tool D, in addition to achieving the above-described effect.

According to at least one embodiment described above, it is possible to support the operator's operation of searching for a medical tool on an ultrasound image.

The term “processor” used in the above explanations denotes, for example, a central processing unit (CPU), a graphics processing unit (GPU), or circuitry such as an application-specific integrated circuit (ASIC) or a programmable logic device (e.g., a simple programmable logic device (SPLD), a complex programmable logic device (CPLD), or a field programmable gate array (FPGA)). The processor reads and executes programs stored in storage circuitry to realize the functions. It is to be noted that the programs may be directly incorporated into the circuitry of the processor, instead of being stored in the storage circuitry. In this case, the processor reads and executes the programs incorporated into the circuitry to realize the functions. On the other hand, if the processor is, for example, an ASIC, the functions are directly incorporated into the circuitry of the processor as logic circuitry, instead of the programs being stored in the storage circuitry. It is also to be noted that each of the processors of the present embodiment is not necessarily configured as a single circuit, and may be configured as a single processor incorporating a plurality of independent circuits realizing the respective functions. Moreover, the constituent elements illustrated in FIGS. 1, 10, 12, and 15 may be integrated into a single processor to realize the respective functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising processing circuitry configured to:
generate a first ultrasound image of a subject into which a medical tool has been inserted by executing, via an ultrasound probe, non-3D scanning of the subject in which at least one scanning surface is formed;
generate a second ultrasound image containing the medical tool by executing 3D scanning; and
estimate, based on the second ultrasound image, an angle of a first scanning surface of an ultrasound probe with which a first ultrasound image containing the medical tool can be generated, wherein
the processing circuitry is configured to switch from the non-3D scanning to execute the 3D scanning during image displaying in the non-3D scanning, and outputs the estimated angle of the first scanning surface.

2. The ultrasound diagnosis apparatus according to claim 1, further comprising a first input interface configured to receive, as an input, an execution instruction to execute the 3D scanning, wherein
the processing circuitry is configured to execute the 3D scanning based on the execution instruction.

3. The ultrasound diagnosis apparatus according to claim 1, wherein the 3D scanning is executed for a single volume.

4. An endocavity ultrasound probe used as the ultrasound probe in the ultrasound diagnosis apparatus according to claim 1.

5. The ultrasound diagnosis apparatus according to claim 1, wherein the processing circuitry is further configured to update position information of the ultrasound probe from an angle of a second scanning surface to the angle of the first scanning surface, the angle of the second scanning surface differing from the angle of the first scanning surface.

6. The ultrasound diagnosis apparatus according to claim 5, wherein
the processing circuitry is configured to perform the updating by changing an angle of a scanning surface from the angle of the second scanning surface to the angle of the first scanning surface.

7. The ultrasound diagnosis apparatus according to claim 5, wherein
the processing circuitry is configured to generate a first ultrasound image containing the medical tool by executing the non-3D scanning via the ultrasound probe whose position information has been updated to the angle of the first scanning surface.

8. The ultrasound diagnosis apparatus according to claim 7, wherein the processing circuitry is further configured to cause a display to display the first ultrasound image containing the medical tool.

9. The ultrasound diagnosis apparatus according to claim 8, wherein the processing circuitry is configured to cause the display to display display screen data in which the first ultrasound image and the second ultrasound image are arranged in parallel.

10. The ultrasound diagnosis apparatus according to claim 1, wherein the processing circuitry is further configured to notify an operator of the angle of the first scanning surface.

11. The ultrasound diagnosis apparatus according to claim 1, wherein the processing circuitry is configured to estimate the angle of the first scanning surface of the ultrasound probe with which the first ultrasound image containing the medical tool can be generated by inputting, to a trained model, the second ultrasound image containing the medical tool generated by the executing of the 3D scanning, the trained model being configured to output the angle of the first scanning surface of the ultrasound probe with which the first ultrasound image containing the medical tool can be generated based on the second ultrasound image containing the medical tool.

12. The ultrasound diagnosis apparatus according to claim 11, wherein
the medical tool includes either a first medical tool or a second medical tool, and
the processing circuitry is configured to estimate an angle of a first scanning surface of an ultrasound probe with which a first ultrasound image containing the first medical tool can be generated in a case of inputting, to the trained model, a second ultrasound image containing the first medical tool, and to estimate the angle of the first scanning surface of an ultrasound probe with which a first ultrasound image containing the second medical tool can be generated in a case of inputting, to the trained model, a second ultrasound image containing the second medical tool.

13. The ultrasound diagnosis apparatus according to claim 11, wherein
the medical tool includes either a first medical tool or a second medical tool,
the trained model includes either a first trained model configured to output an angle of a first scanning surface of an ultrasound probe with which a first ultrasound image containing the first medical tool can be generated based on a second ultrasound image containing the first medical tool, or a second trained model configured to output an angle of a first scanning surface of an ultrasound probe with which a first ultrasound image containing the second medical tool can be generated based on a second ultrasound image containing the second medical tool,
the processing circuitry is further configured to determine, based on the second ultrasound image, whether the medical tool contained by the second ultrasound image is the first medical tool or the second medical tool, and
the processing circuitry is configured to input the second ultrasound image to the first trained model if it has been determined, as a result of the determining, that the medical tool contained by the second ultrasound image is the first medical tool, and to input the second ultrasound image to the second trained model if it has been determined, as a result of the determining, that the medical tool contained by the second ultrasound image is the second medical tool.

14. The ultrasound diagnosis apparatus according to claim 11, wherein
the medical tool includes either a first medical tool or a second medical tool,
the trained model includes either a first trained model configured to output an angle of a first scanning surface of an ultrasound probe with which a first ultrasound image containing the first medical tool can be generated based on a second ultrasound image containing the first medical tool, or a second trained model configured to output an angle of a first scanning surface of an ultrasound probe with which a first ultrasound image containing the second medical tool can be generated based on a second ultrasound image containing the second medical tool,
the ultrasound diagnosis apparatus further includes a second input interface configured to receive, as an input, either a first input instruction to input the second ultrasound image to the first trained model or a second input instruction to input the second ultrasound image to the second trained model, and
the processing circuitry is configured to input the second ultrasound image to the first trained model based on the first input instruction, and to input the second ultrasound image to the second trained model based on the second input instruction.

15. The ultrasound diagnosis apparatus according to claim 1, wherein
the processing circuitry is further configured to detect whether or not the medical tool is contained by the first ultrasound image.

16. The ultrasound diagnosis apparatus according to claim 15, wherein
the processing circuitry is further configured, if it has been detected, as result of the detecting, that the medical tool is not contained by the first ultrasound image, to notify the operator that the medical tool is not contained.

17. The ultrasound diagnosis apparatus according to claim 15, wherein
the processing circuitry is configured to execute the 3D scanning if it has been detected, as a result of the detecting, that the medical tool is not contained by the first ultrasound image.

18. A non-transitory computer readable storage medium including computer executable instructions, wherein the instructions, when executed by a processor, cause the processor to perform a method comprising:
generating a first ultrasound image of a subject into which a medical tool is inserted by executing, via an ultrasound probe, non-3D scanning of the subject in which at least one scanning surface is formed;
generating a second ultrasound image containing the medical tool by executing 3D scanning; and
estimating, based on the second ultrasound image, an angle of the first scanning surface of the ultrasound probe with which a first ultrasound image containing the medical tool can be generated.
